(19) United States
(12) Patent Application Publication
(10) Pub. No.: US 2007/0178537 A1
(43) Pub. Date: Aug. 2, 2007

(54) Assays for detection of bioactive compounds that interact with heat shock protein 90

(75) Inventors: Gabriela Chiosis, New York, NY (US); Neal Rosen, New York, NY (US); Henri Huezo, New York, NY (US)

Correspondence Address: Marina Larson & Associates LLC;re: MSK, P. O. BOX 4928, DILLON, CO, 80435-4928, US

(73) Assignee: Sloan-Kettering Institute for Cancer Research, New York, NY (US)

(21) Appl. No.: 10/595,029

(22) PCT Filed: Jun. 30, 2004
(86) PCT No.: PCT/US04/21297

§ 371 Date: Jun. 9, 2006

Related U.S. Application Data
(60) Provisional application No. 60/483,806, filed on Jun. 30, 2003.

(51) Int. Cl. G01N 33/574; C07K 16/46

(52) U.S. Cl. 435/7.23; 530/391.1

(57) ABSTRACT

A method for evaluation of molecules to identify those that can act as therapeutic inhibitors of Hsp90 makes use of a fluorescence polarization (FP) assay and a cell based assay, either individually or in combination. The FP assay uses a fluorescently-labeled Hsp90 binding agent and measure the degree of fluorescence polarization relative to the standard. A decrease in the degree of polarization indicates that fluorescently-labeled Hsp90 binding agent has been wholly or partially displaced by a candidate molecule, and identifies the molecule as having activity as an inhibitor of Hsp90. The cell based assay tests for decrease is an Hsp90-dependent activity of normal or tumor cells.

### STATEMENT OF RELATED CASES

This application claims the benefit under 35 USC 119(e) of the U.S. Provisional Patent No. 60/483,806 filed Jun. 30, 2003, which is incorporated herein by reference.

### STATEMENT CONCERNING GRANT SUPPORT

The work leading to this invention was supported in part by a grant from NIH-NCI, Grant No. 1U01CA91178-03. The United States may have certain rights in this invention.

### BACKGROUND OF THE INVENTION

This application relates to assay protocols for identification of bioactive compounds that interact with heat shock protein 90.

Heat shock protein 90 (Hsp90) has been identified as a target for therapeutic intervention in a variety of cancers. This intervention can be accomplished using geldanamycin and other ansamycin antibiotics, as well as analogs and derivatives thereof, for example as described in U.S. patent applications Ser. Nos. 09/403,434, 09/937,192 and 09/960,665 which are incorporated herein by reference. Another analog is 17-allylaminogeldanamycin (17-AAG), which is currently being used in human trials. Intervention may also be accomplished using bioactive small molecules which interact with the same binding site on hsp 90 as geldanamycin. (See U.S. patent application Ser. No. 10/415,868, which is incorporated herein by reference). Small molecules are likely to be ultimately preferred for therapeutic applications, since they can avoid some of the difficulties encountered in administration of 17-AAG and other larger compounds.

Her2 is one example of a protein whose expression/stability depends on Hsp90. Identification of compounds that promote Her2 degradation has required cumbersome in vitro analyses involving tissue culture with individual drugs followed by detergent lysis of samples, polyacrylamide gel electrophoresis of cellular proteins, and Western blotting to determine Her2 levels, the methodology being decidedly unsuitable for rapid, high-throughput screening of compound libraries.

Assessment of ligands binding to Hsp90 has also been difficult. Methods included displacement of Hsp90 from immobilized GM, isothermal calorimetry, circular dichroism, measurements of ATPase activity and a filter binding assay using [³H]17AAG. Unfortunately, most of these assays are not suitable for HTS, necessitate high amounts of protein, or depend on radioactive reagents.

### SUMMARY OF THE INVENTION

The present invention provides streamlined assay methods which rely on fluorescence polarization measurements and/or whole-cell immunodetection of Hsp90-dependent proteins to identify bioactive compounds that interact with heat shock protein 90. The first method utilizes minimal amounts of either recombinant Hsp90 or cellular extracts containing Hsp90. The latter method utilizes a minimal number of cells, yet is sufficiently sensitive and reproducible to permit quantitative determinations.

This application provides a method for evaluation of molecules to identify those that can act as therapeutic inhibitors of Hsp90. Such identification presents several challenges. Hsp 90 is a molecular chaperone that is implicated in multiple cell-specific transformation processes, and may play different roles in different cell types. Hsp90 in tumor cells is present in complexes with co-chaperones and transformation-specific client proteins, whereas Hsp90 from normal tissues is more in a latent, uncomplexed state. The constituency of such complexes is dictated by the amount of “stress” on the system (i.e. nature and number of mutated and dysregulated proteins, hypoxia, low nutrient concentration environment). Thus the affinity of an inhibitor for Hsp90 complexes may vary. Assaying for several Hsp90-related events thought to be important in driving transformation is necessary to identify agents that retain a wide-range anti-cancer potential. This application provides several methods for evaluation of molecules to identify those with activity as inhibitors of Hsp90 activity. Each is useful individually. However, they can also be used in sequence, with candidates selected from the first assay procedure being further tested in subsequent assay procedures, to provide maximally efficient identification of compounds.

In a first assay in accordance with the invention, candidate molecules are tested in a competitive assay using fluorescence polarization. In this assay, a candidate molecule is combined with Hsp90 in the presence of a fluorescently-labeled molecule known to bind to Hsp90. The fluorescent label is excited using polarized light and the degree of polarization of the resulting emission is observed. When the fluorescently labeled molecule is bound to the Hsp90, the emission is substantially polarized. On the other hand, when the fluorescently labeled molecule has been wholly or partially displaced by candidate molecule, the extent to which the emission is polarized decreases as a result of the rotational freedom of the unbound fluorescently labeled species.

In a second assay in accordance with the invention, candidate molecules are tested for their ability to bind to Hsp90 by monitoring the ability of the molecule to reduce Hsp90-dependent activity. Preferably, the candidate molecules are ones previously selected using the first assay method of the invention. In this second assay, test cells are grown and preferably attached to a solid support. A compound is added to the test cells, and the cells are incubated for a period of time, for example 6 or 24 hours, sufficient to permit growth of the cells and observable Hsp90-dependent activity in the absence of an effective candidate compound. Thereafter, the amount of Hsp90-dependent activity is determined, for example using an immunological detection system. A reduction in Hsp90-dependent activity is indicative of a candidate molecule with therapeutic potential.

The invention also provides a compound comprising (a) a binding moiety; and (b)a fluorescent moiety; wherein the compound binds, via the binding moiety to Hsp90, and the fluorescent moiety has polarized fluorescence when the compound is bound to Hsp90, and fluorescence with a lesser degree of polarization when the compound is not bound to Hsp90. Examples of such compounds are GM-FITC, GM-BODIPY, and the red shifted GM-BODIPY-TMR.

### BRIEF DESCRIPTION OF THE FIGURES

FIG. 1 shows a synthetic scheme for preparation of GM-BODIPY.

FIG. 2 shows a titration curve of the fluorescence polarization of GM-FITC and GM-BODIPY as a function of Hsp90 alpha concentration.

FIG. 3 shows a saturation curve for GM-BODIPY as different concentrations of Hsp90 alpha. The signal was recorded at the presented time intervals

FIG. 4 shows results of a competition assay using known Hsp90 inhibitors 17AAG and PU3 as compared to Ad-But which is a derivative of PU3 that does not bind to Hsp90.

FIG. 5 shows results of a competition assay using known Hsp90 inhibitors 17AAG and PU24FCL as compared to Ad-But which is a derivative of PU3 that does not bind to Hsp90.

FIG. 6 shows the structures of PU3 and PU24FCL.

FIGS. 7A-C show results of equilibrium binding studies for the FP assay.

FIG. 8 shows results of a study on inhibitory activity of known Hsp90 inhibitors against GM-BODIPY/Hsp90 interaction.

FIGS. 9A and 9B show the effect of DMSO on binding experiments.

FIGS. 10A and 10B show results for tests on a high-throughput format assay.

FIG. 11 shows the difference in binding affinity for tumor versus normal tissue Hsp90.

FIGS. 12A and B show the ability of both recombinant (i.e., purified) Hsp90 alpha, and a cell lysate of SKBr3 to distinguish between compounds with different Hsp90 binding affinities.

FIGS. 13A-C show results for real time biochemical studies using FP assay.

FIG. 14 shows a synthetic scheme for red-shifted BODIPY-TMR-labeled geldanamycin ligand.

FIGS. 15A and B show antibody binding results for SKBr3 cells and A431 cells.

FIGS. 16A and B show results of assay optimization and performance testing using the SKBr3 cells.

FIG. 17 shows the reproducibility of the Her2-blot.

FIGS. 18A and B shows the specificity of the Her2 blot.

FIG. 19 shows the reproducibility of the EGFR blot.

FIG. 20 shows results of the assay test in library format.

FIG. 21 shows the equilibrium between Hsp90 in an activated and in a latent state.

FIGS. 22A-C show results from tests on the use of cell lysates in fluorescence polarization assay.

FIG. 23 shows the antimitotic activity of Hsp90 inhibitos in MDA-MB-468 cells.

FIG. 24 shows Hsp70 assay results. SKBr3 cells were treated with hsp90 inhibitors at 2.5 uM and cellular levels of Hsp70 determined 24 h later using the Hsp70 immunoassay.

### DETAILED DESCRIPTION OF THE INVENTION

This application relates to assays for identification of bioactive compounds that interact with heat shock protein 90.

As used in the specification and claims of this application, the term “assays” refers to test procedures or protocols that when performed provide an indication of the activity of a compound as an inhibitor of Hsp90. The result obtained may be quantitative or qualitative.

As used in the specification and claims of this application, the term “heat shock protein 90” or “Hsp90” when used without modification refers generically to any or all of the different members of the Hsp90 family, including Hsp90 alpha, as reflected in the examples set forth below, Hsp90 beta, grp94 and trap1. The Hsp90 may be in transformation-specific complexes with client proteins, for example as a cellular lysate, or it may be in an uncomplexed form. Uncomplexed Hsp90 is most conveniently available as a recombinant protein. Hsp90 also refers to average population Hsp90 present in cells in transformation-specific complexes. Hsp90 is present in cells in equilibrium between a “latent state” with low chaperoning capacity and an “activated state” with high chaperoning activity (see FIG. 21). The shift in equilibrium may be dictated by the amount of “stress” on the system (i.e. mutated and dysregulated proteins, hypoxia, low nutrient concentration environment). The “activated state” has a higher affinity for ATPase inhibitor drugs than the dynamic “latent state” that is predominant in normal cells. Thus, the effects of inhibiting Hsp90 function may depend more on the “activity” and degree of involvement of the co-chaperone/protein-Hsp90 complexes and less on cellular levels of chaperone. The above data suggest that Hsp90 inhibitor concentrations can be identified that will disrupt critical chaperone functions in transformed cells but that may not be toxic to normal cells, a feature which makes Hsp90 inhibitors attractive as therapeutic agents.

As used in the specification and claims of this application, the term “inhibitor of Hsp90” or “inhibition of Hsp90” refer to either an in vitro or an in vivo reduction in Hsp90 activity. Complete elimination of Hsp90 activity is not required for a molecule to be considered an inhibitor of Hsp90. In general, in vitro reduction in activity of Hsp90 is predicted based upon an ability of a molecule to bind with Hsp90 and displace other binding agents. However, in vivo activity also requires entry of the molecule into the cells and the subsequent binding with Hsp90. Thus, while binding to Hsp90 in vitro is a valid first screening step, it is not a total predictor of the ability of an agent to be an effective therapeutic agent.

The Hsp90 chaperone machinery was found to be required for the correct conformation, function and stability of several kinases, hormone receptors and transcription factors that are directly involved in driving multistep malignancy and of mutated oncogenic proteins required for the transformed phenotype. Hsp90 is also known to be required for the activity of several key regulators of apoptosis, and through these associations the chaperone may confer survival advantage to tumor cells. Inhibition of Hsp90 results in degradation of these client proteins via the ubiquitin proteasome pathway. Hsp90 has been implicated in numerous cellular processes, and in particular in the expression of various cell regulatory proteins and induction of mitotic block and apoptosis in some cells. As used in the specification and claims of this application, the term Hsp90-dependent activity refers to expression of proteins whose expression level in cells is dependent on Hsp90 activity or to the induction of mitotic block (and consequently apoptosis). Non-limiting examples of Hsp90-dependent proteins include Her2, EGFR, Raf-1, Akt, Cdk4, cMet, mutant p53, ER, AR, mutant BRaf, Bcr-Abl and other oncogenic fusion proteins, Flt-3, Polo-1 kinase, HIF-1 alpha, survivin and hTERT.

As used in the specification and claims of this application, the term binding range refers to the difference (maximum mP at saturation−minimal mP at no protein), In an optimized fluorescence polarization assay, the binding affinity of the probe to the protein should be high and the binding range should be large,

All polarization values were expressed in millipolarization units (mP). The mP values were calculated using the equation mP=1000*[(I_{S}−I_{SB})−(I_{P}−I_{PB})]/[(I_{S}−I_{SB})+(I_{P}−I_{PB})], where I_{S} is the parallel emission intensity measurement and I_{P} is the perpendicular emission intensity sample measurement, while I_{SB} and I_{SP} are the corresponding measurements for background (buffer). Total fluorescence was determined as 2*I_{P}+I_{S}. For BODIPY-labeled compounds, measurements were made with excitation at 485 nm (25-nm bandwidth) and emission at 530 nm (25-nm bandwidth) using a 505-nm beam splitter. Specific binding was defined as the contribution to signal of bound ligand and was calculated as b*mPb=mP−f*mPf where b and f are the fractions of bound and free tracer, mP is the recorded polarization value for a particular Hsp90 concentration and mPf if the polarization value for free tracer. The assay window was defined as mP−mPf.

As used in the specification and claims of this application, the term “FITC” refers to fluorescein isothiocyanate. The term BODIPY refers to fluorescent boron dipyridyl compounds.

The first assay of the invention provides a method for identifying a candidate molecule as having activity as an inhibitor of Hsp90 via a competitive binding assay. The method comprises the steps of:
(a) combining the candidate molecule with Hsp90 in the presence of a fluorescently-labeled molecule known to bind to Hsp90;
(b) exciting the fluorescent label using polarized light to produce a fluorescent emission;
(c) observing the degree of polarization of the fluorescent emission; and
(d) comparing the degree of polarization determined in step (c) to a standard value determined for the fluorescently labeled molecule when it is bound to Hsp90. When the fluorescently-labeled molecule is bound to the Hsp90, the emission is substantially polarized. On the other hand, when the fluorescently-labeled molecule has been wholly or partially displaced by candidate molecule, the extent to which the emission is polarized decreases as a result of the rotational freedom of the unbound fluorescently labeled species. Thus, a decrease in the degree of polarization relative to the standard indicates that the fluorescently labeled molecule has been wholly or partially displaced by the candidate molecule, and identifies the molecule as having activity as an inhibitor of Hsp90.

The standard value for the degree of polarization of the fluorescently-labeled molecule can be determined using a separate control reaction conducted in the absence of the candidate compound at the same time as the assay of the candidate compound, or it may be based on a standard or average value determined at a separate time.

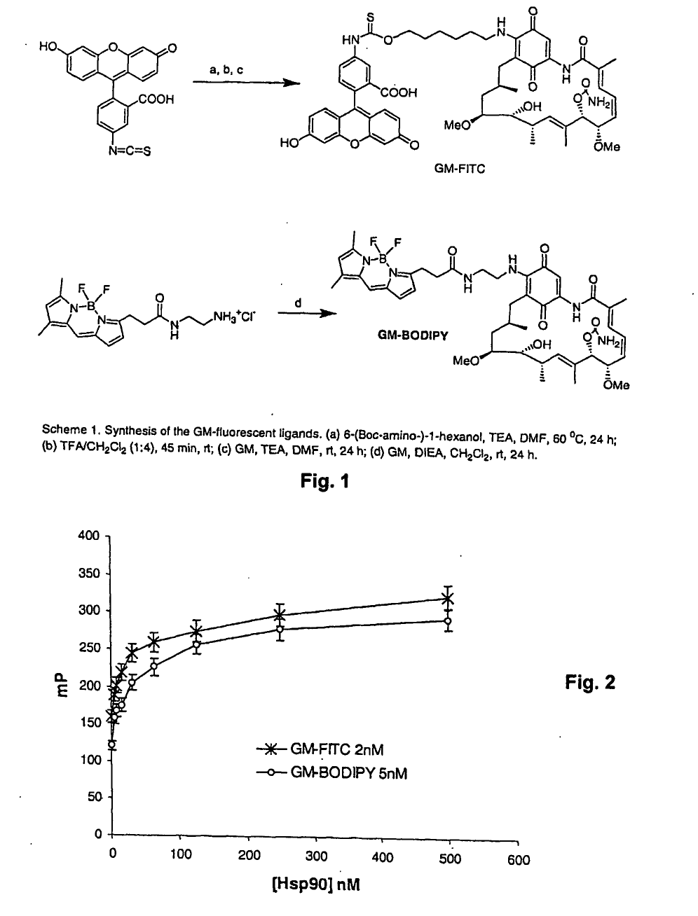

Suitable fluorescently-labeled molecules include those molecules known to bind to Hsp90 which can be modified to incorporate a fluorescent label without interfering with the binding of the molecule to Hsp90. For example, in the case of geldanamycin, modification at C17 to incorporate a fluorescent label such as FITC or BODIPY results in a labeled binding agent which can be used in the assay of the invention. A synthetic scheme for the preparation of such ligands is shown in FIG. 1 and described in Example 1 below. Other suitable fluorescently-labeled molecules can be based on radicicol, and small molecules inhibitors that are discussed above. The amount of Hsp90 employed is sufficient to produce a detectable fluorescence signal. As can be seen in the examples, maximum levels of polarization were observed at Hsp90 alpha concentrations of 50 to 75 nM at constant concentration of the fluorescently-labeled geldanamcyin. While the absolute concentrations may vary from one fluorescently-labeled molecule to another, and from on Hsp90 to another, the assay is desirably performed under conditions, where the degree of polarization observed in the absence of the candidate molecule is substantially independent of the amount of hsp90 present. When using cell lysates instead of recombinant protein, the assay measures binding to average Hsp90 population found in cell specific complexes, be it transformed or normal cell. Suitable cells include all transformed cells in which transformation is driven by an event regulated by hsp90. Specific cell types that are suitably used include, without limitation, mammalian (include human and murine) breast cancer cells (ex. SKBr3, MCF7, MDA-MB-468, BT-474), glioblastoma cells (U87), neuroblastoma cells (ex. SH-SY5Y, N2a), vulvar cancer cells (ex. A431), small cell lung cancer cells (ex. NCI-N417, NCI-H69), prostate cancer cells (ex. LAPC, LNCaP, PC3, TSuPr1), acute myeloid leukemia cells (ex. Kasumi-1, NB4), acute promyelocytic leukemia cells, chronic myeloid leukemia cells (ex. K562), colon cancer cells (ex. Colo205), non-small cell lung cancer cells (ex. A549), melanoma cells (ex. SKMel28), pancreatic cancer cells (ex. AsPC-1, BxPC-3, Capan-2, Miapaca-2, and Panc-1) and normal brain, liver, kidney, pancreas, spleen, lung and heart cells Normal tissue/organs that are suitably used include, without limitation, normal brain, liver, kidney, pancreas, lung and heart cells. Compounds with highest affinity and selectivity for transformed cell hsp90 are favored

The second assay method of the invention of the invention is a cell based assay for identifying a candidate molecule as having activity as an inhibitor of Hsp90. The method comprises the steps of:
(a) adding the candidate molecule to a population of test cells, wherein said test cells have an Hsp90-dependent activity in the absence of the candidate compound;
(b) incubating the cells for a period of time sufficient to permit growth of the cells and exhibit the Hsp90-dependent activity in the absence of an effective candidate compound;
(c) determining the amount of Hsp90-dependent activity; and
(d) comparing the determined amount of Hsp90-dependent activity to a standard value determined for the test cells in the absence of candidate compound, wherein a determined value of the Hsp90-dependent activity that is lower than the standard value by a statistically significant amount is indicative that the candidate molecule has activity as an inhibitor of Hsp90.

The standard value for the Hsp90-dependent activity can be determined using a separate control reaction conducted in the absence of the candidate compound at the same time as the assay of the candidate compound, or it may be based on a standard or average value determined at a separate time.

In one embodiment of the invention the amount of an Hsp90-dependent protein is determined using an immunoassay. One such immunoassay methodology is similar in concept to a western blot assay or a cytoblot assay of the type described by Stockwell et al. Chemistry and Biology 6: 71-83 (1999). These assays, however, test for the presence of a given species. In the second assay, that which is being tested for is the absence or near absence of a chemical species. Testing for the presence of a gene product is far simpler than testing for the absence of a gene product since the amount of gene product presumably increases with time in the first instance, and thus distinguishes itself from any background measurements. In contrast, when the disappearance of a product is measured, the best resolution from background is at time zero, when the assay has not yet started. Surprisingly, notwithstanding this theoretical difficulty, meaningful observations can be made of the loss of Her2 and EGFR expression in the presence of an Hsp90 inhibitor, thus providing an immunoassay which can be used to screen candidtate molecules for activity as inhibitors of Hsp90 and inducers of Her2 and EGFR degradation.

The test cells used in the second assay may be normal cells or tumor cells, provided they exhibit at least one Hsp90-dependent activity. Suitable cells include all transformed cells in which transformation is driven by an event regulated by hsp90. Specific cell types that are suitably used include, without limitation, mammalian (include human and murine) breast cancer cells (ex. SKBr3, MCF7, MDA-MB-468, BT-474), glioblastoma cells (U87), neuroblastoma cells (ex. SH-SY5Y, N2a), vulvar cancer cells (ex. A431), small cell lung cancer cells (ex. NCI-N417, NCI-H69), prostate cancer cells (ex. LAPC, LNCaP, PC3, TSuPr1), acute myeloid leukemia cells (ex. Kasumi-1, NB4), acute promyelocytic leukemia cells, chronic myeloid leukemia cells (ex. K562), colon cancer cells (ex. Colo205), non-small cell lung cancer cells (ex. A549), melanoma cells (ex. SKMel28), pancreatic cancer cells (ex. AsPC-1, BxPC-3, Capan-2, Miapaca-2, and Panc-1) and normal brain, liver, kidney, pancreas, spleen, lung and heart cells Normal tissue/organs that are suitably used include, without limitation, normal brain, liver, kidney, pancreas, lung and heart cells.

The overexpression of Her2 has been associated with aggressive breast malignancies. Overexpression of the receptor tyrosine kinase Her2 in SKBr3 cells leads to Akt activation which in turn promotes cell survival. Hsp90 uniquely stabilizes Her2 via interaction with its kinase domain and an Hsp90 inhibitor induces Her2 degradation by disrupting the Her2/Hsp90 association. Several therapeutic strategies targeting the receptor are now in various stages of clinical development. These strategies act to block the activation or to inhibit the activity of the kinases (i.e. Herceptin and Iressa). ‘Kinase-dead’ receptor can still function as a substrate for other receptor and non-receptor kinases and thus act as a docking protein capable of signaling. Thus, a more significant therapeutic outcome may result from degrading these oncoproteins via Hsp90 inhibition. Like Her2, EGFR is also a transmembrane tyrosine kinase of the HER family (sometimes referred to as Her1) that has been associated with more aggressive clinical behavior of tumors. Accordingly, expression of Her2, EGFR or both are relevant Hsp90-dependent activities which may be tested for in the cell assay of the invention. SKBr3 is used here as an example of Her2 amplification-driven cell, and the assay is not limited to its use. Other cell lines expressing Her2 may be used.

Akt is an important regulator of cell proliferation and survival, and elevated Akt activity has been observed in tumors with mutations in PTEN, one of the most frequently mutated tumor suppressor genes. Mutations in PTEN are found in breast cancer and are associated with poor outcome. PTEN inactivation is frequently found in glioblastomas, melanomas and androgen-independent prostatic adenocarcinomas. Thus, it is reasonable that inducing Akt degradation in these tumors via Hsp90 inhibition may be clinically beneficial. Screening for Akt degradation in U87 (glioblastoma cells, PTEN-defective, high Akt levels/activity) is used to detect agents effective to achieve this result. U87 is used here as an example of PTEN-defective cells that may be used in such assay, and the assay is not limited to its use.

The Raf-MAPK pathway regulates cell proliferation and differentiation and interference with the activity of proteins in this pathway is believed to be effective in cancer treatment. Raf-1 degradation in the MCF7 breast cancer cells (Raf-MAPK driven, high levels of Raf1; Rb+, ER+) is therefore a valid indicator of Hsp90-dependent activity. MCF7 is used here as an example of Raf-MAPK driven cells that may be used in such assay, and the assay is not limited to its use.

Screen for anti-mitotic activity in the Rb-defective breast cancer cell line MDA-MB-468 is also a valid indicator of Hsp90-dependent activity. MA-MB-468 is used here to exemplify Rb defective cancer cell. Other Rb defective tumor cells are small cell lung cancer (SCLC) cells. SCLC is a deadly disease treated primarily with chemotherapy. However, we have reached a plateau with our currently available chemotherapy options and outcomes have not significantly changed over the past 20 years. In order to improve outcomes for these patients, agents that target pathways responsible for tumor development and progression will be required. Nearly all SCLC cell lines and tumors demonstrate functional inactivation of the retinoblastoma gene (RB). This tumor suppressor plays a central role in growth factor induced proliferation by binding to and sequestering the transcription factor E2F. E2F controls the majority of genes required for DNA replication and thus Rb controls cell division by regulating the G1/S transition. As all normal cells in the body express functional Rb, a drug that specifically targets cells with mutationally inactive or deleted Rb would provide a targeted approach for patients with SCLC.

As all normal cells in the body express functional Rb, a drug that specifically targets cells with mutationally inactive or deleted Rb would provide a targeted approach for patients with SCLC. One can identify such agents by assaying for anti-mitotic activity first in an Rb-defective cell (ex. MDA-MB-468) and further in an Rb positive cell (ex. MCF7). These assays will weed out agents that block cells in mitosis by other mechanisms, such as microtubule interacting agents.

In performing immunoassays assays of the second type of assay, various parameters are important to achieve the best performance. These include antibody optimization, cell number and blocking and washing buffers. The antibodies used in the examples below were selected based on general criteria and the procedures set forth below.

For Her2 antibody optimization, we screened a panel of primary and secondary antibodies searching for a reasonable signal to noise ratio. We found two combinations of primary and secondary antibodies, the anti Her2 SC 284, a rabbit polyclonal antibody that binds to the carboxy terminus region of the protein, with the anti rabbit SC 2004 (both from Santa Cruz Biotechnologies), and the anti Her2, #28-004 (Zymed Laboratories) with the anti-rabbit HRP-linked antibody (Sigma, A-0545) to perform excellently in the Her2 blot assay. The signal resulting from specific antibody binding was significantly greater than nonspecific antibody binding to the plate or cells. For EGFR, the best signal was obtained with the rabbit polyclonal antibody #06-847 from Upstate Biotechnologies. Interestingly, the best performing antibodies in the Her2- and EGFR-blot also work well in western blot assays.

To assess cell number optimization, experiments were performed with SKBr3, a breast cancer cell line that expresses high levels of Her2 and A431, a vulvar cancer line with EGFR overexpression. As expected, the signal in untreated cells increased with increasing cell number, however, the ratio of signal over background (S/B) reached a plateau at approximately 8,000 plated cells/well. A significant S/B value was obtained using a minimum 1000 cells/well (S/B=20), however the calculated Z′ value (Zhang, et al., “A simple statistical parameter for use in evaluation and validation of high throughput screening assays. J. Biomol. Screening 4, 67-73, 1999) at this cell number was of a borderline value (Z=0.46). Cell plating numbers above 1000 cells/well were considered useful in a screening assay, the recorded Z′ values and S/B ratios being higher than 0.5 or at least 25, respectively, with satisfactory plating a number of approx. 3,000 cells/well for SKBr3 and approx. 2,000 cells/well for A431 being achieved. A time interval of 48 h between plating and assay was used to attain a good attachment and confluency of the cells in the well.

In our hands, Adequate S/B ratios (>30) were obtained using Corning black, clear bottom plates and Perkin Elmer white, clear bottom plates. Paradoxically, the Corning white, clear bottom plates gave higher background readings and although the signal was additionally high, the maximum S/B value was only 14. Thus preference was given to Corning black, clear bottom plates and all data presented here were recorded in such plates.

Assay test optimization involves consideration of various statistical parameters and other factors.

1. Z′ Values

A good signal window is essential for hit identification in a HTS assay. Variations associated with vehicle only treated cells and background IgG measurements are taken in account by the Z′ coefficient. The Z′-parameter is a characteristic of the assay itself, without intervention of compounds. The variability in the obtained signal of the Her2-blot is due to the fact that all reagents were manually added, and would likely be diminished with automatization. Even with the introduction of human error, a Z′ value of 0.7 is obtained, value considered excellent for a screening assay.

2. Reproducibility of Data

To test the practicability of our conditions and the accuracy of the Her2/EGFR-blot, we tested several agents whose action on Her2 and EGFR was either reported in literature or analyzed in our group by immunoblotting (western blot). It was determined that addition of an Hsp90 inhibitor to SKBr3 cancer cells induced the rapid proteasomal degradation of Her2, most protein being depleted at 6 h. Degradation of EGFR occurs with different kinetics; considerable effects on EGFR in the A431 cell line are observed only at 12 h. To observe maximal protein depletion, a 24 h drug treatment was considered more appropriate. The time frame, however, can be set to an interval that best suits the purpose of the screening assay.

IC50 values for Her2 degradation obtained by our method were compared to western blot analysis results. The Hsp90 inhibitor, PU24FCl, and the natural products GM and RD gave values in the Her2-blot method identical to the western blot assay. Addition of PU24FCl to SKBr3 cells induces the degradation of Her2 with an IC50 value of ˜4 M. The values obtained with GM and RD were 17 nM and 28 nM, respectively, consistent with prior literature reports. The assay was also performed after a 6 h drug treatment, to again reproduce data obtained by western blot.

To rule out that the declining signal in drug treated cells was not the result of reduced cell number caused by unspecific cell death but to decreased Her2 content, we determined the amount of total protein by the bicinchoninic assay (BCA) (Smith, et al. “Measurement of protein using bicinchoninic acid”. Anal. Biochem. 50, 76-85, 19985) and additionally, tested changes in β-actin protein levels. We found the BCA assay to be compatible with the Her2-blot reagents. Two 96-well plates were treated with various concentrations of PU24FCl; the first was subjected to a BCA assay alone, and the second to a Her2-blot experiment followed by a BCA assay. Measured differences in total protein between the two plates were insignificant and an average of 1.5-1.6 g protein was detected in each well. We additionally monitored possible changes in β-actin levels due to drug addition, prior to and following the Her2-blot. If the β-actin-blot was performed subsequent to the Her2-blot, plates were stripped and blocked for nonspecific binding prior to the addition of an anti-actin antibody. As expected, the levels of actin in the stripped plate were slightly diminished but detectable. Thus this method, similar to western blot, allows for re-blotting of the plate. No significant differences in actin expression were seen between untreated and Her2-depleted cells validating the observation that the lower Her2 levels in treated cells were indeed due to a selective reduction in oncoprotein levels. If, however, variation in total protein content is observed, values obtained for Her2 can be normalized to total protein concentration. Both the BCA assay and -actin-blot are compatible with the Her2-blot and can be performed following Her2 quantification. It is noteworthy that actin-level measurements could be indicative of the antiproliferative effect of the tested compounds. Thus in addition to identifying changes in Her2, screening would be indicative of cytotoxic effects of the compounds.

3. Stability of the Assay

The Her2-blot assay signal is stable and the obtained values reproducible from plate-to-plate and day-to-day measurements. PU24FCl was used as a test compound on several plates at different time periods. The curves obtained in separate readings of this compound were overlapping (IC50s of 4.00, 4.18 and 4.54; standard deviation of 6%).

PU24FCl was also tested in the A431 cell line to determine the feasibility of the assay in quantifying EGFR depletion. Using western blot and EGFR-blot an IC50 value for EGFR degradation of approximately 25 M was observed. As the Her2-blot, the EGFR-blot was robust, with data obtained by the method reproducible over several plates.

The assay method of the second type can also be used as a library screening format as reflected below in the examples. The technique has advantages over traditional western blot methods in that it is faster and less labor intensive, and therefore better suited to screening of large numbers of the candidate molecules.

While in its simplest form the second type of assay can be performed using a single transformation-specific cellular “fingerprint” of Hsp90 inhibition, its is desirable to use multiple transformation types to provide a more detailed cellular “fingerprint” of Hsp90 inhibition which can show a wider scope of therapeutic applicability for a given candidate molecule. To provide such a detailed fingerprint, test cells are suitably selected to provide information about diverse Hsp90-dependent activities. Thus, in one embodiment of the invention, a candidate molecule is screened using any combination of two or more of the following cell types, and preferably all four:
(1) cells with Her2 driven transformation, as exemplified by SKBr3 breast cancer cells that are high Her2 driven, Rb⁺ cells; determine level of Her2;
(2) cells with Raf-MAPK driven transformation, as exemplified by MCF7 breast cancer cells that are Rb⁺, ER⁺ cells with high levels of Raf1; determine level of Raf-1
(3) cells which are PTEN-defective cells with high Akt levels/activity, as exemplified by U87 glioblastoma; determine level of Akt
(4) cells which are Rb⁻, as exemplified by MBA-MD-468 breast cancer cells which are Rb⁻, ER⁻, determine induction of mitotic block and apoptosis.

An important benefit of the second assay as disclosed in this application is that it is not dependent on the specific mechanism by which the agent acts. Rather, the assay provides a blinded screening for agents that down-regulate kinase expression via action on transcription or translation and/or affect RNA or protein half-life. The microtiter plate format is further beneficial because it requires expenditure of minimal amounts of unknown compound, thus making this an ideal platform for small molecule library screening. This non-biased assay is capable of detecting compounds that affect transcription, translation or stability of Her2 or other Hsp90-dependent proteins. Use of the assay for this purpose is relevant because, (1) if the currently available agents for inhibition are not sufficiently potent, administering several inhibitors that work by different mechanisms might combine to cause sufficient signaling blockade; (2) inhibitors of expression would not leave kinase-dead receptor in the membrane as docking proteins; and (3) in many tumors, increased HER-kinase activity is due to gene amplification or overexpression by other mechanisms.

Running solely the FP binding assay will identify compounds that may be of high affinity but have poor cell permeability profiles. It could also generate “false-positives.” On the other hand, running solely the cell-based assay is more time consuming and more costly. Thus, screening for Hsp90 binding in the FP assay and then testing identified candidate compounds in the cell based assay that “reads” a cellular fingerprint of Hsp90 inhibition is a preferred embodiment for the invention.

Hsp90 inhibition modulates a stress response. Stress-induced synthesis of Hsp70 is regulated at the transcriptional level via the activation of heat shock transcription factors (HSF). Hsp70 levels are induced by Hsp90 inhibitors due to disruption of the HSF1-Hsp90 complex. One may use Hsp70 induction by Hsp90 inhibitors as a potential cellular read-out of Hsp90 dependent activity. For this purpose we have developed a microtiter assay that “reads” cellular levels of Hsp70, which is described below.

In a further aspect, the present invention provides compounds that can be used in the fluorescence polarization assay of the invention. The term “compound” is used in its ordinary chemical meaning to refer to a pure substance composed of two or more elements whose composition is constant. These compounds comprise (a) a binding moiety; and (b) a fluorescent moiety. The binding moiety and the fluorescent moiety are covalently associated although the compound may be in the form of a salt. The compound binds, via the binding moiety, to Hsp90 in the ATP/ADP binding pocket. The fluorescent moiety has polarized fluorescence when the compound is bound to Hsp90, and fluorescence with a lesser degree of polarization (including no polarization) when the compound is not bound to Hsp90. By way of non-limiting example, the compounds of the invention may include FITC, BODIPY or red-shifted BODIPY as the fluorescent moiety and geldanamycin, herbimycin, radicicol, or other Hsp90-binding species as described above as the binding moiety. Specific examples of compounds in accordance with the invention are GM-FITC, GM-BODIPY, and GM-BODPY-TMR.

The invention will now be further described with reference to the following non-limiting examples.

### EXAMPLES

### Example 1

### Synthesis of Fluorescent-Labeled Geldanamycin

The sole known chemical modification in the skeleton of GM allowing for activity is at C17 (Schnur et al. “Inhibition of the Oncogene Product p185erbB-2 in Vitro and in Vivo by Geldanamycin and Dihydrogeldanamycin Derivatives”, J. Med. Chem. 1995, 38, 3806-3812; Schnur et al. “erbB-2 Oncogene Inhibition by Geldanamycin Derivatives: Synthesis, Mechanism of Action, and Structure-Activity Relationships”, J. Med. Chem. 1996, 38, 3813-3820.). The methoxy group found at this position easily undergoes a Michael reaction when in the presence of primary amines. Two fluorescent dyes primarily used in FP assays, FITC and BODIPY, were chosen to be linked to GM. The commercially available fluorescein-5-isothiocyanate was reacted with 6-(Boc-amino)-1-hexanol by heating in DMF in the presence of triethylamine (TEA) as base to afford the corresponding thiocarbamate in 50% yield. Deprotection of Boc using trifluoroacetic acid (TFA) in CH2Cl2 occurred in 45 min and resulted in the corresponding TFA salt. This was reacted with GM in DMF in the presence of TEA for 24 h. The reaction afforded GM-FITC in 60% yield. For the construction of GM-BODIPY we used the commercially available 4,4-difluoro-5,7-dimethyl-4-bora-3a,4a-diaza-s-indacene-3-propionyl ethylenediamine hydrochloride (BODIPY® FL EDA) (Molecular probes # D2390). The Michael reaction of this derivative with GM occurred smoothly in CH2Cl2 with diisopropylamine (DIEA) as base to afford GM-BODIPY. The linker between GM and dyes is longer than 6 atoms and should not considerably obstruct binding to the chaperone. The specific reaction conditions are as follows.

GM-FITC: To a solution of FITC-NCS (78 mg, 0.20 mmol) and 6-(Boc-amino)-1-hexanol (43 mg, 0.20 mmol) in DMF (0.75 mL) at 60° C. was added TEA (16 L, 0.4 mmol). After it was heated with stirring for 24 h, the solution was cooled to room temperature, and the solvent was removed in vacuo. Silica gel column chromatography with DCM/hexanes/MeOH (6:4:1) afforded the corresponding thiocarbamate (60 mg, 50%) as an orange crystalline solid. 1H NMR (400 MHz, MeOH-d4): 7.33-7.25 (m, 6H), 7.21-7.16 (m, 4H), 5.76-5.68 (m, 1H), 5.05 (d, J=17.1 Hz, 1H), 5.01 (d, J=10.2 Hz, 1H), 4.51-4.48 (m, 1H), 4.05 (dd, J=1.6, 9.0 Hz, 1H), 3.98 (t, J=8.4 Hz, 1H), 3.44-3.36 (m, 2H), 3.25 (dd, J=3.1, 14.0 Hz, 1H), 3.20 (dd, J=3.3, 13.4 Hz, 1H), 2.66 (dd, J=9.8, 13.4 Hz, 1H), 2.48-2.44 (m, 2H). MS m/z 608.1 (M+H). To the thiocarbamate (7 mg, 0.012 mmol) was added a mixture of CH2Cl2/TFA (0.3 mL/0.1 mL) and the resulting solution was stirred at room temperature for 45 min. The solvent was removed in vacuo. The crude was taken up in DMF (0.5 mL) and GM (6.4 mg, 0.012 mmol) and an excess triethylamine (50 L, 36 mmol) were added to the solution. The mixture was stirred under inert gas atmosphere for 24 h. After solvent removal in vacuo, the product was purified on a silica gel column eluting with CH2Cl2/EtOAc/hexanes/MeOH (4:2:3:1) to afford GM-FITC (5.5 mg, 44%) as a yellow-orange solid.

GM-BODIPY: A solution of BODIPY® FL EDA (1.1 mg, 0.003 mmol), GM (2.5 mg, 0.005 mmol) and DIEA (3 L, 0.018 mmol) in CH2Cl2 (0.6 mL) was stirred for 24 h. The mixture was added to a silica gel column and eluted with DCM/acetone (3:1) to afford GM-BODIPY (1.5 mg, 60%) as an orange solid.

### Example 2

### Fluorescence Polarization of GM-FITC and GM-BODIPY

To assess the suitability of these probes for Hsp90 in a homogenous FP assay format using an Analyst AD (Molecular Devices) instrument, a stock of 10 M of each tracer was prepared in DMSO and diluted with HFB buffer (20 mM HEPES (K) pH 7.3, 50 mM KCl, 1 mM DTT, 5 mM MgCl2, 20 mM Na2MoO4, 0.01% NP40 with 0.1 mg/mL BGG) to obtain 10 nM and 4 nM solutions for GM-BODIPY and GM-FITC, respectively. Different amounts of Hsp90 alpha (Stressgen # SPP776) dissolved in HBF were added to a low binding black 96 well-plate (Corning # 3650) in a 50 L volume. To each well were added 50 L of the tracer solution. Some wells were left with buffer or tracer alone to serve as controls. The plate was left on a shaker at 4° C. for 3 h and the FP values in mP were recorded. The measured FP value (mP) was plotted against the protein concentration (FIG. 2). Both tracers performed well in the assay. The titration curve showed that the probes bind tightly to Hsp90 The dynamic binding range of FP was approximately 160 mP.

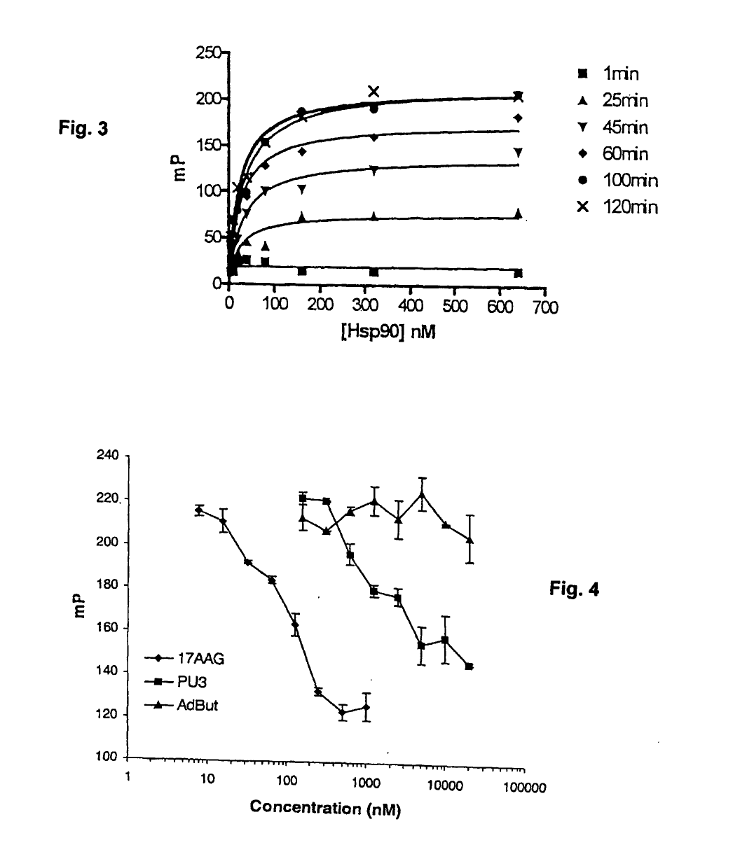

FIG. 3 shows a saturation curve for binding of 4 nM GM-BODIPY to hsp alpha. At lower Hsp90 concentrations, a low mP value was obtained for the fluorescent ligand. As the concentration of the Hsp90 alpha was increased, a greater fraction of fluorescent GM-BODIPY is bound to the protein, and polarization progressively increases to reach saturation. The GM-BODIPY tracer has a Kd of 6.6 nM and a maximum binding range of 160 mP.

### Example 3

### Competitive Displacement of GM-BODIPY by Hsp90 Inhibitors

Competitive displacement studies were performed with known Hsp90 inhibitors 17AAG and PU3 (structure shown in FIG. 6) and additionally as a control, with Ad-But, a PU3 derivative that does not bind Hsp90 (Schulte, et al. “The benzoquinone ansamycin 17-allylamino-17-demethoxygeldanamycin binds to Hsp90 and shares important biologic activities with geldanamycin.” Cancer Chemother. Pharmacol., 42: 273-279, 1998; Chiosis, et al., “A small molecule designed to bind to the adenine nucleotide pocket of Hsp90 causes Her2 degradation and the growth arrest and differentiation of breast cancer cells.” Chem. Biol., 8: 289-299, 2001). Stocks of these molecules were made in DMSO at concentrations of 200 M for 17AAG and 4 mM for PU3 and Ad-But. The drugs were serially diluted in binding buffer and the GM-BODIPY tracer and Hsp90 were added at 5 nM and 40 nM concentrations, respectively. Maximum concentration of used DMSO was 0.25% (v/v). The plate was left on a shaker at 4° C. for 5 h and the FP values in mP were recorded. A window of 100 mP was observed between wells containing protein and tracer and wells containing tracer only. The measured FP values (mP) were plotted against the competitor concentration (FIG. 4). EC50 values were determined as the competitor concentrations were 50% of the tracer was displaced. As previously reported, PU3 was found to be a 30-times weaker Hsp90 inhibitor than 17AAG (EC50=3.2 M vs 110 nM). Ad-But could not displace Hsp90 bound GM-BODIPY even at the maximally measured concentration of 20 μM.

### Example 4

### Competitive Displacement of GM-BODIPY by Hsp90 Inhibitors

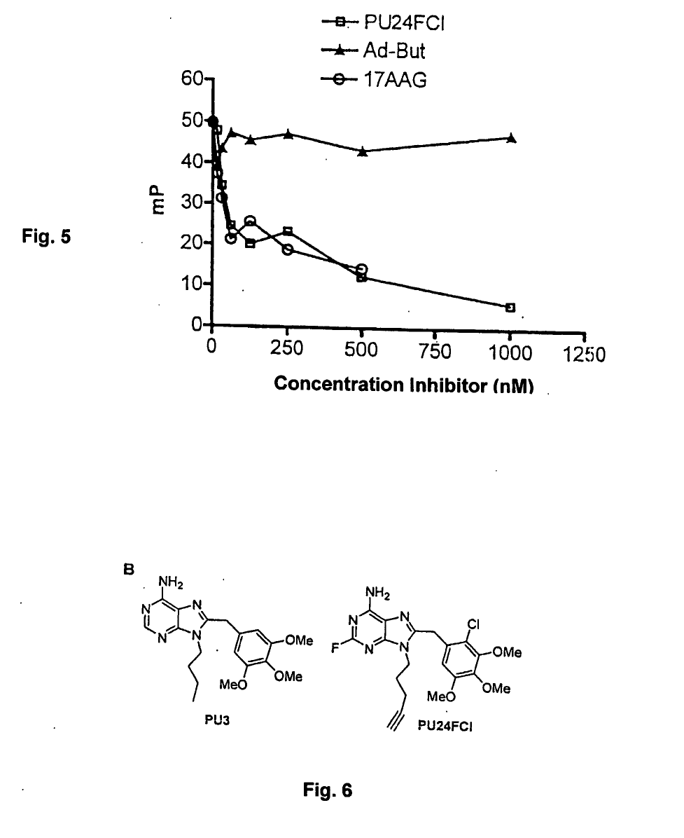

Example 3 was repeated using 30 nM Hsp90 alpha, 4 nM GM-BODIPY to test known Hsp90 inhibitors PU24FCL (structure shown in FIG. 6) and 17AAG. Ad-But was again used as a control. The results are shown in FIG. 5. As shown, PU24FCL have similar activity, while the control compound shows no activity. It should be noted that the binding range differs in this example from others in this application because the assay conditions were not optimized.

### Example 5

### Equilibrium Binding Analysis of the Assay

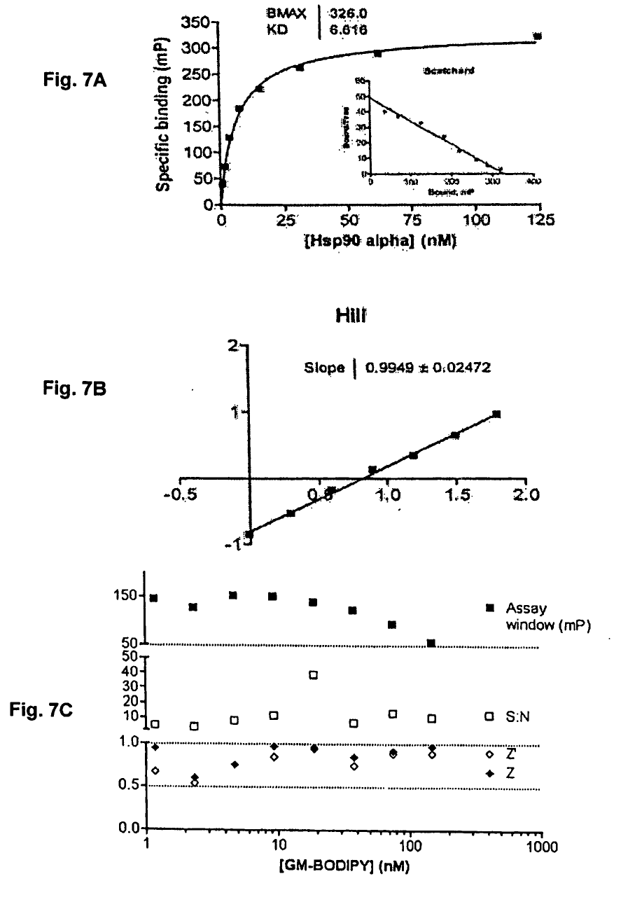

Different amounts of recombinant Hsp90 protein (0-125 nM) were incubated with 5 nM GM-BODIPY at 4° C. and the response measured. Fluorescence polarization was read with an Analyst AD instrument. Polarization values obtained were plotted against Hsp90 concentrations. Specific binding represents the contribution of bound ligand to total recorded values and was calculated as b*mPb=mP−f*mPf where b and f are the fractions of bound and free tracer, mP is the recorded polarization value for a particular Hsp90 concentration and mPf if the polarization value for free tracer. Data were analyzed in Microsoft®Excel and plotted in Prism 4.0. Data collected at equilibrium in the binding experiment were transformed and analyzed using a non-linear regression method in Prism 4.0. (FIGS. 7A and B). Scatchard and Hill plots were constructed to demonstrate one-site binding. (FIG. 7B). The binding of 1-150 nM GM-BODIPY to 30 nM Hsp90 was measured in the presence (displaced) and absence (bound) of 0.5 M GM. The polarization signal for tracer alone in buffer (free) was also measured. Data recorded were used to determine the assay window, Z, Z′ and S:N values and these were plotted as a function of GM-BODIPY concentration. (FIG. 7C).

### Example 6

### Inhibitory Activity of Known Hsp90 Inhibitors Against GM-BODIPY/Hsp90 Interaction

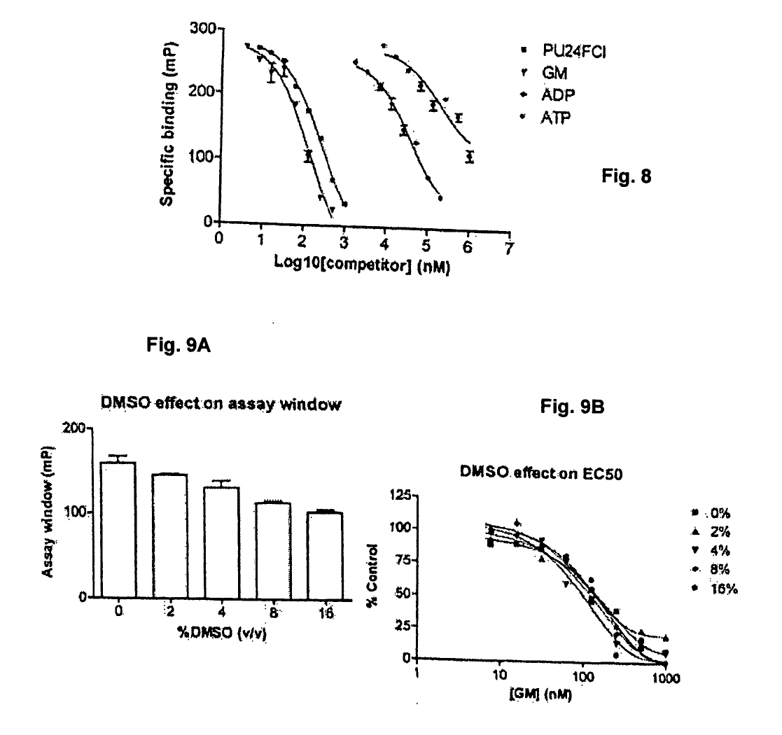

GM, PU24FCl, ATP and ADP were serially diluted in buffer on a 96-well plate. A mixture of 30 nM Hsp90 and 5 nM GM-BODIPY was added and the plate incubated at 4° C. Specific ligand binding was plotted against the log10 inhibitor concentration and EC50 values calculated using a non-linear least square curve fitting program in Prism 4.0. The results are shown in FIG. 8. All compounds were used as DMSO stocks.

### Example 7

### Effect of DMSO on Binding Experiments

Competitive binding experiments of GM with added 0, 2, 4, 8 and 16% DMSO (v/v) were performed in the presence of 5 nM fluorescent GM and 30 nM Hsp90 . Data from the binding results were analyzed using Prism 4.0. (FIG. 9A) Data recorded in tracer only wells were subtracted from control wells (no GM present) and plotted as a function of DMSO concentration to present the effect of the organic solvent on the assay window. The assay window was defined as mP−mPf. (FIG. 9B) Values recorded in wells with added GM were normalized to data in control wells and plotted against the concentration of tested GM for each DMSO concentration.

### Example 8

### High-Throughput Format Assay Evaluation

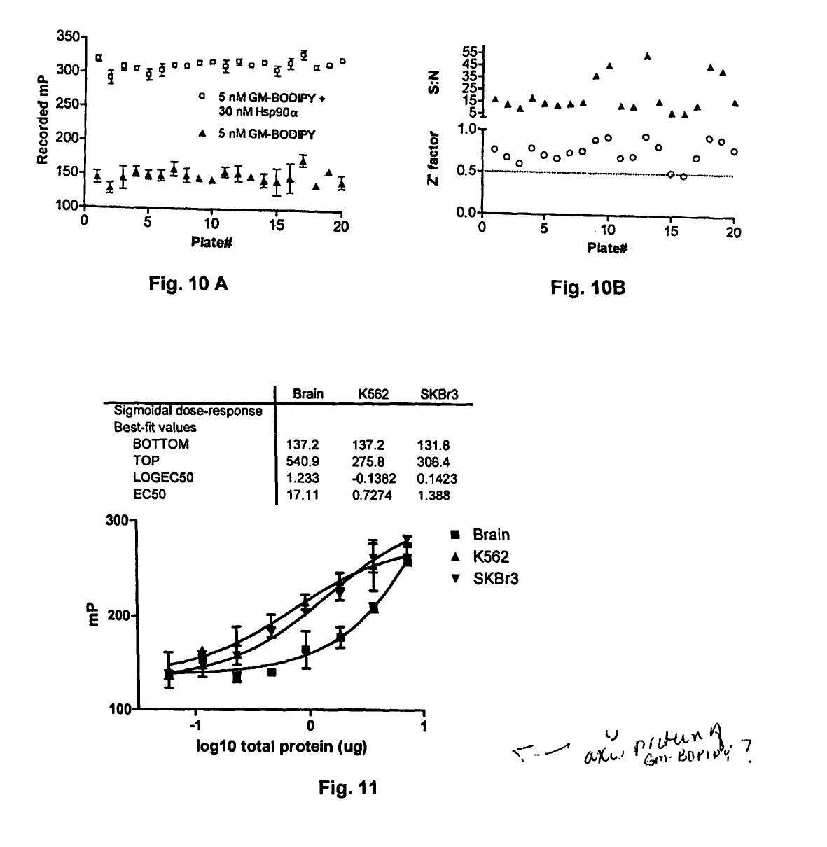

Twenty 96-well plates each containing four free tracer control wells (5 nM GM-BODIPY) and four bound tracer control wells (5 nM GM-BODIPY with added 30 nM Hsp90) were used to determine the suitability of the assay for HTS (all other wells contained compounds tested for binding to Hsp90). Each plate corresponds to an assay conducted on a different day. The mP value for each well was recorded and average values corresponding to each plate were plotted. (FIG. 10A) The signal-to-noise ratios and the Z′ factors were calculated for each plate. (FIG. 10B)

### Example 9

### Fluorescence Polarization Assay Using Cellular Lysates

An important issue in screening assays is the cost of reagents. Although our assay uses very low amounts of recombinant Hsp90 protein, the ability to use the assay with Hsp90 from cell lysate would expand its usefulness and reduce its cost. In addition, as it is believed that tumor Hsp90 is present entirely in multi-chaperone complexes with high ATPase activity, whereas Hsp90 from normal tissues is in a latent, uncomplexed state, it would be more therapeutically significant to measure affinity to Hsp90 in its cellular state. Hsp90 plays a role in maintaining the function of conformationally labile signal transducers that act in growth control, cell survival and tissue development. Post-translational interaction with these targets allows Hsp90 to link the cell to its environment and couple the stress response to integrated changes in multiple signal transduction pathways. Under normal conditions, the dynamic low-affinity interactions of Hsp90 with client proteins serve to maintain them in a latent state that is capable of activation. Upon mutation or dysregulated function many of these client proteins display unusually stable associations with Hsp90-containing chaperone complexes and these associations appear to be essential for their transforming, aberrant activity. Thus, it is likely that many Hsp90-client proteins are transformation-specific (cell specific) clients. The affinity of these Hsp90-complexes may also be cell type specific. These may be reflective of differential binding affinity of certain Hsp90 inhibitors to Hsp90 in cell-type specific complexes, difference that may be overlooked by screening for binding to recombinant Hsp90. Hsp90 is highly expressed in stressed cells, thus small amounts of total protein might be necessary for its detection in cell lysates.

For use in the assay of the invention, cell lysates are made using the following protocol
collect cells
adherent cells: trypsinize (or scrape), harvest cells in 15 ml conical tubes and centrifuge at 1,600 RPM for 5 minutes at 2 to 8 C
(use confluent T-175 flask to obtain 0.5 ml of lysate)
floating cells: pellet cells by centrifugation 1600 rpm for 5 minutes at 2 to 8 C
   wash 2× with 5 ml of PBS buffer
   resuspend the pellet in 1 ml of PBS buffer and transfer cells to an eppendorf tube
   centrifuge, aspirate supernatant, and freeze the pellet O/N at −70° C.
   resuspend in lysis buffer B (Felts buffer) (1×106 cells per 100 l of buffer B)
   incubate on the rotor for 2 hours at 2 to 8° C.
   centrifuge the samples at 13,200 RPM for 30 min at 2 to 8° C.
   transfer the supernatant to a fresh tube
   quantitate protein concentration with the Pierce BCA assay
   add 0.5 uL of 1M DTT/mL lysate
   store samples at −70 C;
      before conducting an experiment thaw samples on ice and keep samples on ice throughout the course of experiment.

To prepare lysis buffer B, add the following to 5 ml of Felts Buffer (Felts buffer: 20 mM HEPES (K) pH 7.3, 50 mM KCl, 5 mM MgCl2, 20 mM Na2MoO4, 0.01% NP40):

| | |
|---|---|
| 20 l | Na3VO4 (1 mM) |
| 25 l | Pefabloc SC (50 g/ml) |
| 7.5 l | pepstatin (15 g/ml) |
| 7.5 l | leupeptin (15 g/ml) |
| 7.5 l | aprotinin (15 g/ml) |

If tissue/tumors are used instead of cultured cells, these are homogenized directly in the binding buffer.

Cellular lysates were prepared in this manner from normal brain tissue, CML cell line K562 and breast cancer cell line SKBr3. For constructing a saturation curve, 5 nM GM BODIPY was added to the lysate, and the fluorescence polarization assay of the invention was performed. As shown in FIG. 11, there is a difference in binding affinity for tumor versus normal tissue Hsp90 that can be observed in the assay.

For competitive binding using cell lysates, a typical assay consists in incubating the lysate (the necessary amount is determined from saturation curve data, as in FIG. 13C for MCF7) with 5 nM GM BODIPY and varying amounts of inhibitors and reading the polarization signal with an Analyst AD plate reader. An example of competitive assay using MCF7 lysate is presented in FIG. 22B.

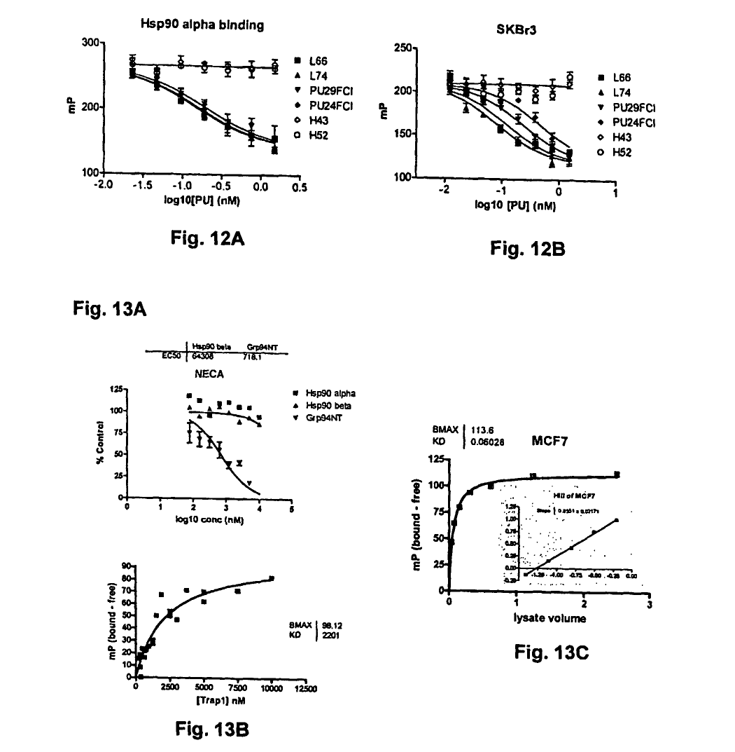

FIGS. 12A and B show the ability of both recombinant (i.e., purified) Hsp90 alpha, and a cell lysate of SKBr3 to distinguish between compounds with different Hsp90 binding affinities. The x axis is the concentration of the specified compound. H43 and H52 are used as controls with no Hsp90 binding affinity. Both assay systems provide the necessary discrimination.

GM-BODIPY binds well to cellular Hsp90 in lysates resulting in an assay window of 110 mP in MCF7 breast cancer cell line (FIG. 13C). The Hill plot analysis of this binding experiment shows that the low amounts of lysate required (˜1-3 ug total protein) prevent unspecific interaction from other cellular material (Hill curve slope of 1).

The assay can measure affinity of compounds for hsp90 from tumor and normal cells. As shown in FIG. 22C, there is a difference in binding affinity of PU24FCl for tumor hsp90 versus normal tissue Hsp90 that can be observed in the assay.

### Example 10

### Real Time Biochemical Studies Using FP Assay

FP assay is useful in determining “real-time” interactions with Hsp90 family members, selectivity for Hsp90 family members (see NECA a Grp94 specific inhibitor does not inhibit the cytosolic Hsp90 a and b) and binding to Trap-1, the mitochondrial Hsp90; and affinity for Hsp90 in tumor cell lysates.

Cells of interest are frozen to rupture membranes and then dissolved in binding buffer to form the lysate used in further analyses. If tissue/tumors are used, these are homogenized directly in the binding buffer. A typical assay consists in incubating the lysate (the necessary amount is determined from saturation curve data, FIG. 13C or 22A for MCF7) with 5 nM GM-BODIPY and varying amounts of inhibitors and reading the polarization signal with an Analyst AD plate reader.

NECA was serially diluted in buffer on a 96 well plate. A mixture of 30 nM Hsp90 alpha, 30 nM Hsp90 beta or 200 nM Grp94-His, respectively with 5 nM GM BODIPY was added and the plate incubated at 4EC for 7 h. Specific ligand binding was plotted against the log10 inhibitor concentration and EC50 values calculated using a non linear least square curve fitting program in Prism 4.0. The results are shown in FIG. 13A.

Different amounts of recombinant Trap-1 protein (0 10 microM) were incubated with 5 nM GM BODIPY at 4° C. and the response measured. Fluorescence polarization was read with an Analyst AD instrument. Polarization values obtained were plotted against trap-1 concentrations. The results are shown in FIG. 13B.

### Example 11

### Synthesis of Red-Shifted BODIPY-TMR-Labeled GM

One concern in FP assays is compound interference. This interference can be due to fluorescent compounds or insoluble compounds in the libraries. The two main factors that can modulate such interference are the concentration and the emission wavelength of the fluorophore. When the fluorophore is used at low concentrations, fluorescence contribution from library compounds may have an impact on the assay because the detection readout is a measure of the sum of all fluorescent species in the assay. As the fluorophore concentration is increased, the contribution from other fluorescent compounds is reduced. Use of BODIPY-FL with an emission of 530 nM increases the likelihood of generating false positives. We have not detected such interference in the screening of our purine-skeleton library of Hsp90 inhibitors. However, some LOPAC™ compounds such as dipyridamole and quinacrine, described by the Schering-Plough team as the highest autofluorescent compounds of that library, interfered with readings at low GM-BODIPY concentrations.

One way around this problem would be using higher concentrations of GM-BODIPY. However, that may also require the use of higher concentrations of competitor in the screens. In addition, the assay window decreases as higher fluorophore concentrations are used (50 mP at 150 nM GM-BODIPY). A better way around this issue, would be to use a red-shifted label, such as BODIPY-TMR in labeling of GM as fewer compounds emit at such long wavelength. In addition, this might help reduce light scattering caused by insoluble compounds.

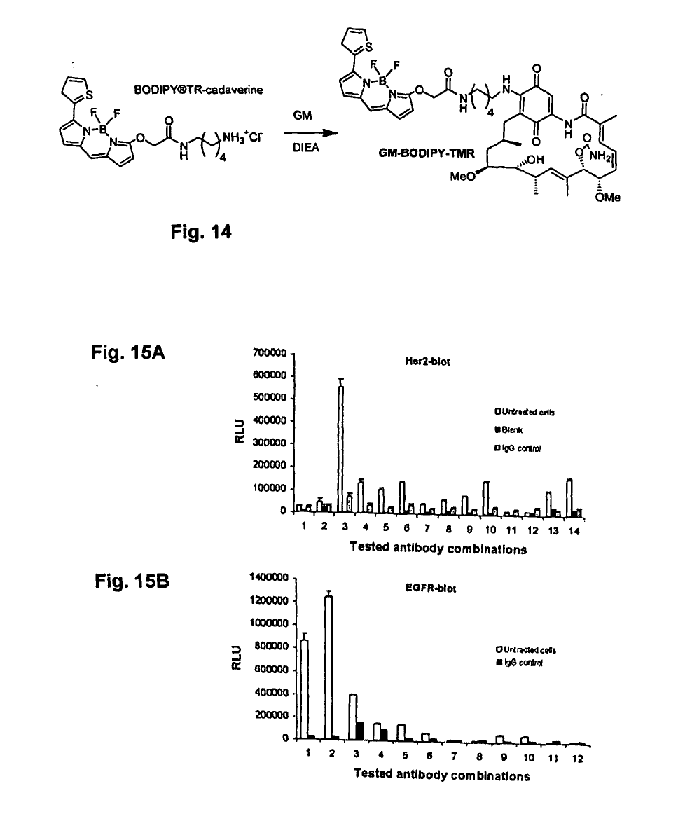

Synthesis of the red-shifted BODIPY-TMR-labeled geldanamycin ligand can be accomplished using the reaction scheme shown in FIG. 14. 5-(((4-(4,4-difluoro-5-(2-thienyl)-4-bora-3a,4a-diaza-s-indacene-3-yl) phenoxy)acetyl)amino)pentylamine, hydrochloride (BODIPY® TR cadaverine, Molecular Probes) is coupled with GM in CH2Cl2 with diisopropylamine (DIEA) as base to afford GM-BODIPY-TMR using the basic procedures of Example 1.

### Example 12

### Screening of Antibodies for Best Signal to Noise Ratio

The human cancer cell lines SKBr3 and A431 were obtained from the American Type Culture Collection (Manassas, Va.) and maintained in 1:1 mixture of DME:F12 supplemented with 2 mM glutamine, 50 units/mL penicillin, 50 units/mL streptomycin and 5% heat inactivated fetal bovine serum (FBS) (Gemini Bioproducts) and incubated at 37° C. in 5% CO2. Stock culture was grown in T-175 flasks containing 30 mL of DME (HG, F-12, non-essential amino acids, and penicillin and streptomycin), with glutamine, and 10% FBS. Cells were dissociated with 0.05% trypsin and 0.02% EDTA in phosphate buffer saline (PBS) without calcium and magnesium.

SKBr3 cells and A431 cells were treated for 24 h with vehicle. Cells were fixed and permeabilized and the Her2-blot or EGFR-blot reagents added. The results are summarized in FIGS. 15A and B, respectively. Each column represents the luminescence reading obtained from the combination of a primary and a secondary antibody. Columns: 1=Ab1+Sec1, 2Ab2+Sec2, 3=Ab2+Sec1, 4=Ab2+Sec2 etc. For Her2: Ab1=Transduction Laboratories# E19420, Mouse IgG2b, epitope: N-terminal domain; Ab2=Santa Cruz#SC-284, rabbit polyclonal, epitope: carboxy terminus; Ab3=Oncogene#OP39, mouse IgG1, epitope: extracellular domain; Ab4=NeoMarkers#MS-267, Mouse monoclonal, epitope: extracellular domain; Ab 5=NeoMarkers#MS-301, mouse monoclonal, epitope: extracellular domain; Ab6=Oncogene#OP16, Mouse IgG2a, epitope: extracellular domain; Ab7=NeoMarkers#MS-599, epitope: C-terminus. For EGFR: Ab1=Upstate Biotechnologies#06-847, rabbit polyclonal IgG, epitope: C-terminal domain; Ab2=Upstate Biotechnologies#06-129, sheep polyclonal IgG, epitope: cytoplasmic domain; Ab3=Upstate Biotechnologies#05-104, mouse monoclonal IgG, epitope: Ala351-Asp364; Ab4=BD Pharmigen, mouse IgG; Ab5=NeoMarkers#MS-269-P1, mouse monoclonal, epitope: extracellular domain; Ab6=Calbiochem#PC19, rabbit polyclonal IgG. When Ab is rabbit, Sec1=Santa Cruz #SC-2004 and Sec2=Sigma #A0545. When Ab is mouse, Sec1=Amersham#NXA931 and Sec2=Sigma#A9044. When Ab is sheep, Sec1=Sigma (#A3415) and Sec2=Upstate Biotechnologies (#12-342). Blank is a measure of unspecific binding of the primary and secondary antibodies to the plate. IgG control represents the reading obtained by addition to cells of a normal IgG and the corresponding HRP-linked secondary antibody.

### Example 13

### Assay for Her2 Expression

Experimental cultures of SKBr3 were plated in black, clear-bottom microtiter plates (Corning # 3603) (3,000 cells per well) in growth medium (100 L), and allowed to attach for at least 48 h at 37° C. and 5% CO2. Some wells were left without cells to serve as the blank. Growth medium (100 μL) with drug or vehicle (DMSO) was carefully added to the wells and the microtiter plates were placed at 37° C. and 5% CO2. DMSO levels should not exceed 0.1% as higher levels were found to affect the growth of cancer cells.

Following incubation (6 h or 24 h), wells were washed twice with ice-cold tris buffer saline (TBS) containing 0.1% Tween20 (TBST) (200 μL). A house vacuum source attached to an 8-channel aspirator was used to remove the liquid from the microplates. Further, methanol (100 μL at −20° C.) was added to each well and the plate was left at 4° C. for 10 min. Methanol was removed by washing with TBST (2×200 μL). The plate was further incubated at RT for 1 h with SuperBlock® (Pierce # 37535) (200 μL) and overnight at 4° C. with the anti-Her-2 antibody (Santa Cruz Biotechnology # SC-284) (100 L, 1:200 in SuperBlock®). Each well was washed with TBST (2×200 μL) and incubated at RT for 2 h with an anti-rabbit HRP-linked antibody (Sigma # A0545) (100 μL, 1:1000 in SuperBlock®). Unreacted antibody was removed by washing with TBST (3×200 μL) and the chemiluminescent substrate solution (100 L) (Pierce # 38040) was added. The plate was read 5 min later in an Analyst AD plate reader (Molecular Devices). Each well was scanned for 0.1 s. Readings from wells containing only control IgG and the corresponding HRP-linked secondary antibody were set as background and deducted from all measured values. Luminescence readings resulted from drug treated cells vs untreated cells (vehicle treated) were quantified and plotted against drug concentration to give the IC50 values (defined as concentration of drug required to degrade 50% of total Her2).

Other anti-Her2 antibodies tested were purchased from Transduction Laboratories (#E19420), NeoMarkers (#MS-267, MS-301, MS-599), Oncogene (#OP16, OP39). Another anti-rabbit-HRP antibody was purchased from Santa Cruz Biotechnology (# CS-2004) and the anti-mouse-HRP antibodies from Amersham (#NXA931) and Sigma (#A9044). Normal rabbit and mouse IgGs were purchased from Santa Cruz Biotechnology (# SC-2027 and SC2025).

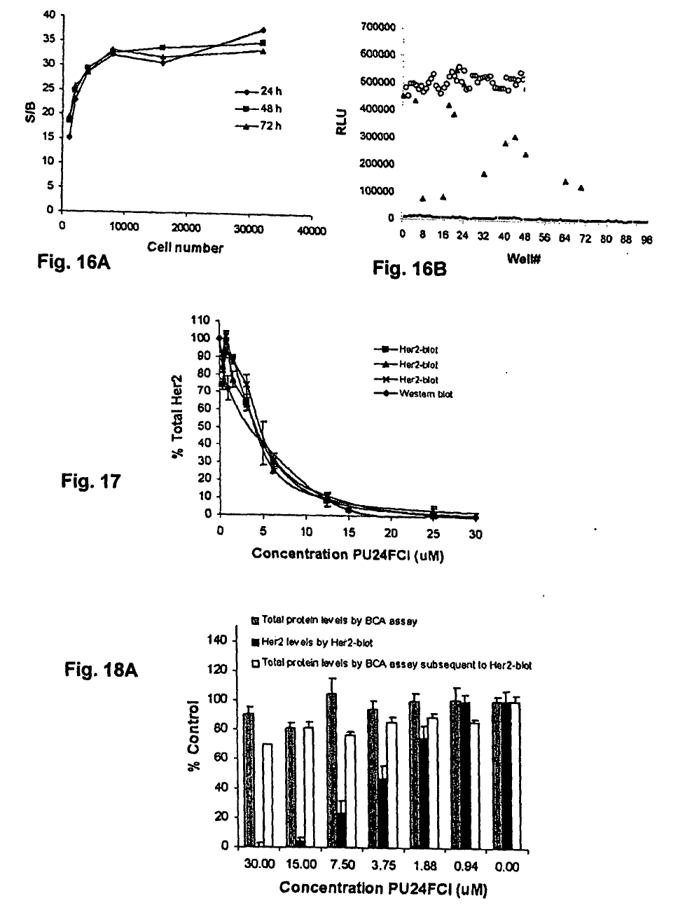

FIGS. 16A and B show results of assay optimization and performance testing using the SKBr3 cells. For evaluate the optimal cell number, SKBr3 cells were left to attach for the indicated time period prior to Her2-blot analysis. The signal recorded in anti-Her2 (S) and IgG (B) reacted wells was recorded and the ration S/B was plotted as a function of cell number (FIG. 6A). Each measurement represents the average of 8 wells. Results of Z′-analysis in the Her2 blot are shown in FIG. 16B. SKBr3 cells were reacted with the anti-Her2 antibody (open circles; std=7.5%), the corresponding IgG (solid squares; std=16.8%) and twenty Hsp90 inhibitors [at 30 M] from an in-house library (solid triangles).

FIG. 17 shows the reproducibility of the Her2-blot. SKBr3 cells were treated with various concentrations of PU24FCl for 24 h in 10-cm plates for western blot and 96-well plates for Her2-blot. Her2 content was quantified by each method and plotted vs drug concentration. Values obtained from three Her2-blot experiments conducted on various plates and time intervals are plotted (P<0.1). All values were normalized for total protein content. The western blot data represents the average of three experiments.

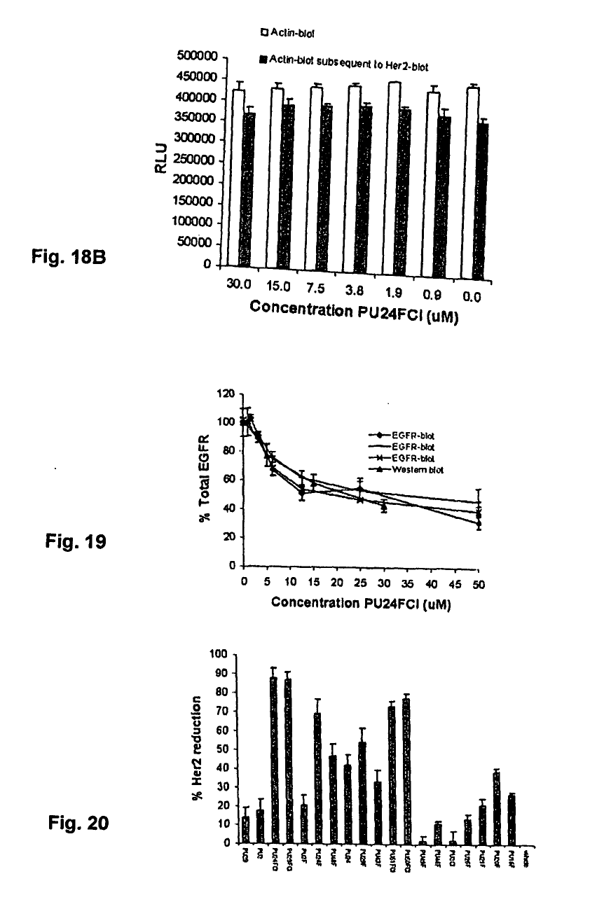

FIGS. 18A and B shows the specificity of the Her2 blot. SKBr3 cells were treated for 24 h in two 96-well plates with either vehicle or increasing concentrations of PU24FCl. Cells were fixed and permeabilized. Total protein concentration was determined using the BCA assay: without prior manipulations in the first plate (FIG. 18A, gray bars) and subsequently to the Her2-blot in the second plate (FIG. 18A, white bars). Black bars depict Her2 protein levels determined by the Her2-blot. Each measurement is an average of 6-wells. Control=vehicle only treated cells. Changes in β-actin levels were determined upon addition of drug: without prior manipulations in the first plate (FIG. 18B, white bars) and subsequently to the Her2-blot in the second plate (FIG. 18B, gray bars).

### Example 14

### Cytoblot Assay for EGFR Expression

An EGFR-blot experiment was conducted identically as for Her2. The anti-EGFR antibody used was purchased from Upstate Biotechnology (#06-847) and was used 1:500 in SuperBlock®. Other anti-EGFR antibodies tested were purchased from Upstate Biotechnology (#06-129, 05-104), Santa Cruz Biotechnology (#SC-03-G), NeoMarkers (#MS-269-P1), BD Pharmigen (#610016) and Calbiochem (#PC19). The anti-sheep HRP-linked antibodies and the normal sheep IgG were purchased from Sigma (#A3415), Upstate Biotechnologies (#12-342) and Santa Cruz Biotechnology (#SC2717), respectively.

FIG. 19 shows the reproducibility of the EGFR blot. A431 cells were treated with various concentrations of PU24FCl for 24 h in 10-cm plates for western blot and 96-well plates for EGFR-blot. EGFR content was quantified by each method and plotted vs drug concentration. Values obtained from three EGFR-blot experiments conducted on various plates and time intervals are plotted (P<0.1). All values were normalized for total protein content. The western blot data represents the average of three experiments. As with the Her2 test, the assay was robust, and data obtained by the method was reproducible across several plates.

### Example 15

### Assay Test in Library Format

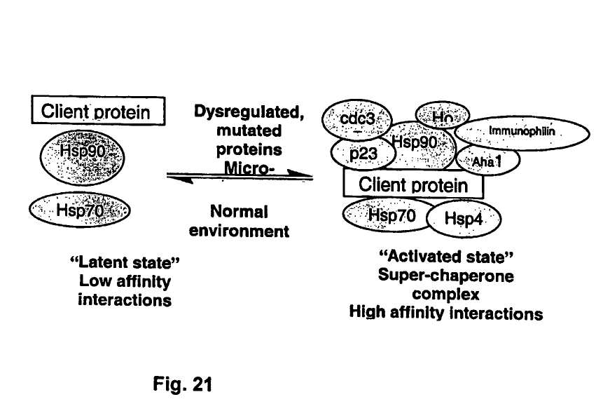

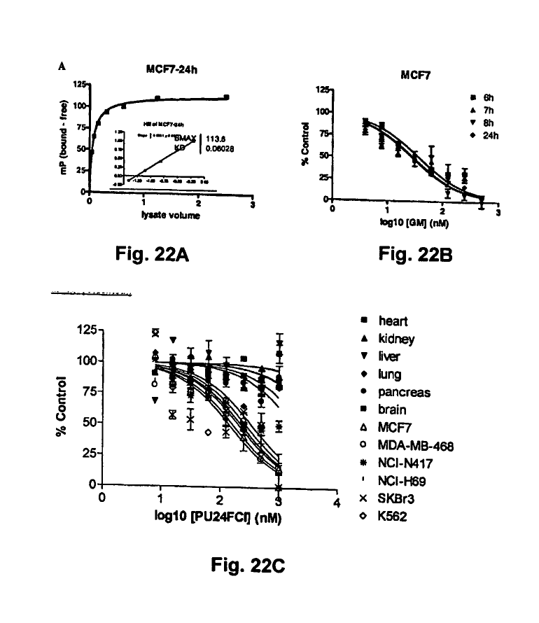

To confirm the utility of the assay in a library screen format to identify agents capable of altering the cellular levels of these kinases, a subset of our Hsp90-inhibitor library was added to SKBr3 cells at a set concentration of 30 μM and the ability of these agents to decrease Her2 levels in cells was determined after a 6 h treatment. Agents that alter cellular levels of kinase by at least 25-30% can be considered “hits” with a high degree of confidence. There was no significant change in total protein content as determined by the BCA assay and thus, the values reflect the effect of these compounds on Her2 expression. The result are summarized in FIG. 20. The average signal obtained for each of these agents (in RLU) is plotted as the solid triangles in FIG. 16B. It is noteworthy to mention that identical activity in the series of compounds was previously obtained using the traditional western blot, however this work required a 3-week period.

### Example 16

### Use of Cell Lysates in Fluorescence Polarization Assay

Fluorescence polarization measurements were performed on an Analyst AD instrument (Molecular Devices, Sunnyvale, Calif.). Measurements were taken in black 96-well microtiter plates (Corning # 3650). The assay buffer (HFB) contained 20 mM HEPES (K) pH 7.3, 50 mM KCl, 5 mM MgCl2, 20 mM Na2MoO4, 0.01% NP40. Before each use, 0.1 mg/mL bovine gamma globulin (BGG) (Panvera Corporation, Madison, Wis.) and 2 mM DTT (Fisher Biotech, Fair Lawn, N.J.) were freshly added. Cell lysates were prepared rupturing cellular membranes by freezing at −700 C and dissolving the cellular extract in HFB with added protease and phosphotase inhibitors. Organs were harvested from a healthy mouse and homogenized in HFB. Saturation curves were recorded in which GM-BODIPY (5 nM) was treated with increasing amounts of cellular lysates. The amount of lysate that resulted in polarization (mP) readings corresponding to 20 nM recombinant Hsp90 was chosen for the competition study. For the competition studies, each 96-well contained 5 nM fluorescent GM, cellular lysate (amounts as determined above and normalized to total Hsp90 as determined by Western blot analysis using as standard Hsp90 purified from HeLa cells (Stressgen# SPP-770)) and tested inhibitor (initial stock in DMSO) in a final volume of 100 L. For liver, the amount of GM-BODIPY had to be increased due to high autofluorescence of the liver homogenate. The plate was left on a shaker at 4° C. for 7 hr and the FP values in mP were recorded. EC50 values were determined as the competitor concentrations were 50% of the fluorescent GM was displaced.

FIG. 22A is a saturation curve determined by adding increasing volumes of cellular lysate to 5 nM GM-BODIPY. The Hill plot (slope of 1) suggests one-site binding at these amounts of total protein, thus specific Hsp90 interaction. FIG. 22B shows a competitive binding curve obtained by incubation of MCF7 lysate (vol. determined from A, when 99% ligand is bound) with GM-BODIPY 5 nM and increasing [GM]. The assay is stable and results in reproducible data (6-24 h readings presented). The apparent affinity of PU24FCl for Hsp90 in several cell lysates was examined by a fluorescence polarization (FP) method that measures the ability of the agent to compete fluorescently labeled GM for Hsp90 binding. Normal organs (heart, kidney, liver, lung, pancreas and brain) and several transformed cells (breast cancer cells MCF7, SKBr3 and MDA-MB-468, small cell lung cancer cells NCI-N417 and H69, and chronic myeloid leukemia cell line K562) are presented. The results are summarized in FIG. 22C.

### Example 17

### Protocol for Determination of Anti-Mitotic Activity

Black, clear-bottom microtiter 96-well plates (Corning Costar 3603) were used to accommodate experimental cultures. 8000 MDA-MB-468 cells per well were seeded in each well with 100 μL of growth medium, and allowed to attach for 24 hours at 37 C and 5% CO2. After 24 hours of the initial seeding, growth medium (100 μL) with drug or vehicle (DMSO) was gently added to the wells, and the plates were plated at 37 C and 5% CO2 for another 24 hours.

Following the 24-hour incubation, the wells were washed twice with ice-cold 1×TBST (Tris Buffer Saline containing 0.1% Tween 20). A house vacuum source attached to an eight-channle aspirator was used to remove the liquid from the 96-well plates. Then, 100 μL of ice-cold methanol was added to each well, then placed in 4 C for 5 min. After 5 min, methanol was suctioned out, and the plates were washed three times with ice-cold 1×TBST (2×200 μL). After the removal of methanol, the plates were further incubated with SuperBlock® blocking buffer (Pierce #37535) for 2 hours at RT. Then TG3 antibody was diluted 1:200 in SuperBlock®, and placed in each well (100 μL) except the first column; the first column was treated with Ig control (Neg. Control for Mouse IgM, NeoMarkers, NC-1030-P), 1:200 dilution in Superblock®. After 72 hours, all wells were washed with ice-cold 1×TBST twice, then secondary antibody (Goat Anti-Mouse IgM, SouthernBiotech, 1020-05) in 1:2000 dilution in SuperBlock® was placed in each well, and incubated on a shaker at RT for 2 hours.

Un-reacted antibody was removed by washing the plates with ice-cold 1×TBST for 5 min on a shaker three times. Then 100 μL of ECLTM Western Blotting Detection Reagents 1 and 2 in 1:1 mix was placed, column by column with 8-channel pipetter with 5 sec delay to compensate for the speed of machine readings. The plates were read immediately in an Analyst AD plate reader (Molecular Devices).

Luminescence readings were then imported into SOFTmax PRO® 4.3.1 DD. Readings from wells treated with DMSO was used as background to be subtracted from all measured values, and the graph of RLU versus Concentration was plotted.

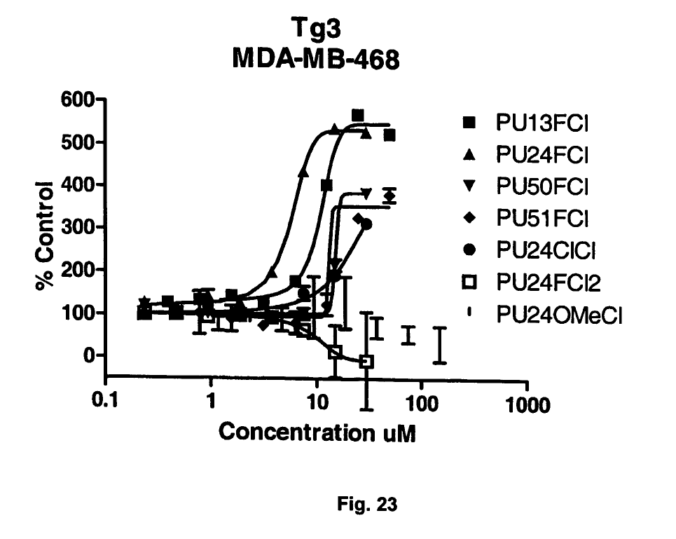

FIG. 23 shows the antimitotic activity of Hsp90 inhibitos in MDA-MB-468 cells. As shown MDA-MB-468 cells were treated for 24 h with several small molecules. The percentile increase in cells in mitosis compared to untreated cells was quantified using a p-nucleolin specific antibody.

### Example 18

### Hsp70 Assay

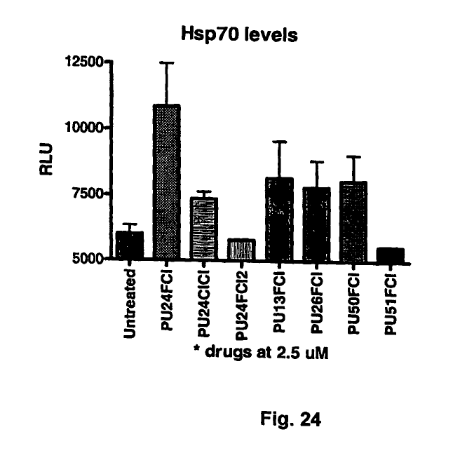

On a white, clear bottom 96-well plate (Packard#6005181), 6000 SkBr3 cells were plated in each well in 100 μL of medium, and the plate was incubated at 37° C. and 5% CO2 for 24 h. After this attachment period, 100 μL of growth medium containing drug (final concentration of 2.5 uM) or vehicle (DMSO) was pipetted gently on top of the existing 100 μL medium, and the plate was incubated for an additional 24 h. Each drug was added in quadruplicate. Following the 24-hour incubation, 2×200 μL of ice-cold TBST (Tris Buffer Saline+0.1% Tween 20) was used to wash the plate. Cells were fixed and permeabilized by the addition of 100 μL MeOH (−20 C) for 10 min at 4 C. Methanol was removed by washing the plate three times with ice-cold TBST. Plates were blocked with 200 μL of SuperBlock® (Pierce#37535) for 90 min. The anti-HSP70 antibody (Stressgen# SPA-810), 100 L at 1:500 dilution in SuperBlock® was placed in each well. Control wells were added 100 μL of normal mouse IgG (NeoMarker) at the same dilution with SuperBlock®. The plate was left in the cold room overnight, and then washed with TBST (3×200 μL). The secondary antibody (100 μL) (Amersham, Sheep Anti-Mouse IgG HRP # NXA931) at 1:2000 dilution in 5% BSA was added to all the wells for 2 hours. Un-reacted antibody was removed by washing three times with ice-cold TBST for 5 min on a shaker. ECL reagent (100 μL) was added and plates were read immediately with an Analyst AD plate reader (Molecular Devices). Luminescence readings were then imported into SOFTmax PRO® 4.01. Readings from IgG Control wells were used as background to be subtracted from all measured values. The results of this experiment are shown in FIG. 24.

1. A method for identifying a candidate molecule as having activity as an inhibitor of Hsp90 comprising the steps of:
(a) combining the candidate molecule with Hsp90 in the presence of a fluorescently-labeled molecule known to bind to Hsp90;
(b) exciting the fluorescently-labeled molecule using polarized light to produce a fluorescent emission;
(c) observing the degree of polarization of the fluorescent emission; and
(d) comparing the degree of polarization determined in step (c) to a standard value determined for the fluorescently labeled molecule when it is bound to Hsp90, wherein a decrease in the degree of polarization relative to the standard indicates that the fluorescently labeled molecule has been wholly or partially displaced by the candidate molecule, and identifies the molecule as having activity as an inhibitor of Hsp90.

2. The method of claim 1, wherein the Hsp90 is provided in the form of a cell lysate.

3. The method of claim 1, wherein the candidate molecule is further tested , if it is identified the molecule as having activity as an inhibitor of Hsp90, by a second assay comprising the steps of:
(a) adding the candidate molecule to a population of test cells, wherein said test cells exhibit an Hsp90-dependent activity in the absence of the candidate compound;
(b) incubating the cells for a period of time sufficient to permit growth of the cells and exhibit the Hsp90-dependent activity in the absence of an effective inhibitor;
(c) determining the amount of the Hsp90-dependent activity; and
(d) comparing the determined amount of Hsp90-dependent activity to a standard value determined for the test cells in the absence of an effective inhibitor, wherein a determined value of the Hsp90-dependent activity that is lower than the standard value by a statistically significant amount is indicative that the candidate molecule has activity as an in vivo inhibitor of Hsp90.

4. The method of claim 3, wherein the test cells are tumor cells.

5. The method of claim 4, wherein the test cells are selected from the group consisting of breast cancer cells, glioblastoma cells, neuroblastoma cells, vulvar cancer cells, small cell lung cancer cells, prostate cancer cells, acute myeloid leukemia cells, acute promyelocytic leukemia cells, chronic myeloid leukemia cells, colon cancer cells, non-small cell lung cancer cells, melanoma cells, and pancreatic cancer cells.

6. The method of claim 3, wherein in the second assay the candidate molecule is added to a plurality of populations of different cell type, wherein the different cell types have at least partially different Hsp90-dependent activity.

7. The method of claim 6, wherein the different cell types includes cells selected from the group consisting of high Her2 driven cells; Raf-MAPK driven cells; PTEN-defective cells with high Akt levels/activity; and Rb-defective cells.

8. The method of claim 6, wherein different cell types include two or more cell types selected from the group consisting of
(1) SKBr3 breast cancer cells;
(2) MCF7 breast cancer cells;
(3) U87 glioblastoma cells; and
(4) MBA-MD-468 breast cancer cells.

9. The method of claim 1, wherein the fluorescently-labeled molecule is a labeled geldanamycin labeled at the C17 position.

10. The method of claim 1, wherein the Hsp90 is Hsp90 alpha.

11. The method of claim 1, wherein the Hsp90 is Hsp90 beta.

12. The method of claim 1, wherein the Hsp90 is grp94.

13. The method of claim 1, wherein the Hsp90 is trap1.

14. The method of claim 1, wherein the fluorescently-labeled molecule comprises a FITC label.

15. The method of claim 1, wherein the fluorescently-labeled molecule comprises a BODIPY label.

16. The method of claim 1, wherein the fluorescently-labeled molecule comprises a red-shifted BODIPY label.

17. The method of claim 1, wherein the amount of fluorescently-labeled molecule is such that the degree of polarization observed in the absence of the candidate molecule is substantially independent of the amount of Hsp90 present.

18. A method for identifying a candidate molecule as having activity as an inhibitor of Hsp90, comprising the steps of
(a) adding the candidate molecule to a population of test cells, wherein said test cells exhibit an Hsp90-dependent activity in the absence of the candidate compound;
(b) incubating the cells for a period of time sufficient to permit growth of the cells and exhibit the Hsp90-dependent activity in the absence of an effective inhibitor;
(c) determining the amount of the Hsp90-dependent activity; and
(d) comparing the determined amount of Hsp90-dependent activity to a standard value determined for the test cells in the absence of an effective inhibitor, wherein a determined value of the Hsp90-dependent activity that is lower than the standard value by a statistically significant amount is indicative that the candidate molecule has activity as an in vivo inhibitor of Hsp90.

19. The method of claim 18, wherein the test cells are tumor cells.

20. The method of claim 19, wherein the test cells are selected from the group consisting of breast cancer cells, glioblastoma cells, neuroblastoma cells, vulvar cancer cells, small cell lung cancer cells, prostate cancer cells, acute myeloid leukemia cells, acute promyelocytic leukemia cells, chronic myeloid leukemia cells, colon cancer cells, non-small cell lung cancer cells, melanoma cells, and pancreatic cancer cells.

21. The method of claim 18, wherein a second assay is performed in which the candidate molecule is added to a plurality of populations of different cell type, wherein the different cell types have at least partially different Hsp90-dependent activity.

22. The method of claim 21, wherein the different cell types includes cells selected from the group consisting of high Her2 driven cells; Raf-MAPK driven cells; PTEN-defective cells with high Akt levels/activity; and Rb-defective cells.

23. The method of claim 21, wherein different cell types include two or more cell types selected from the group consisting of
(1) SKBr3 breast cancer cells;
(2) MCF7 breast cancer cells;
(3) U87 glioblastoma cells; and
(4) MBA-MD-468 breast cancer cells.

24. A compound comprising
(a) a binding moiety; and
(b) a fluorescent moiety;
wherein the compound binds, via the binding moiety to Hsp90, and the fluorescent moiety has polarized fluorescence when the compound is bound to Hsp90, and fluorescence with a lesser degree of polarization when the compound is not bound to Hsp90.

25. A compound according to claim 22, wherein the fluorescent moiety is a BODIPY.

26. The compound according to claim 22, wherein the fluorescent moiety is a red-shifted BODIPY.

27. The compound according to claim 22, wherein the fluorescent moiety is FITC.

28. The compound according to claim 22, wherein the binding moiety is geldanamycin.

29. The compound according to claim 22, having the structure: GM-BODIPY

30. The compound according to claim 22, having the structure: GM-BODIPY-TMR

31. The compound according to claim 22, having the structure: GM-FITC

32. A method of claim 1, wherein the fluorescently-labeled molecule comprises a binding moiety and a fluorescent moiety, and wherein the molecule via the binding moiety to Hsp90, and the fluorescent moiety has polarized fluorescence when the compound is bound to Hsp90, and fluorescence with a lesser degree of polarization when the compound is not bound to Hsp90.

33. A method according to claim 32, wherein the fluorescent moiety is a BODIPY.

34. A method according to claim 32, wherein the fluorescent moiety is a red shifted BODIPY.

35. A method according to claim 32, wherein the fluorescent moiety is FITC.

36. A method according to any of claim 32, wherein the binding moiety is geldanamycin.
